SEQUENCE LISTING (3 sequences)

SEQ ID NO: 1 - DNA, 344 bp, Artificial Sequence
  source: /note=“Description of artificial sequence Synthetic polynucleotide”
gtnnnaanttacggagggncctcagattnagtnctaggaaagagctggtaccattggggatggccacaatgattctagcgtgcgtgtcgtcaggttggcgccttgaagtgcctcaagatccttggagcaggaggagcagcagcagcagcagcagcagcagcagcagcagcagcagcagcagcagcagcagcagcagcagcagcagcaggagcagcagcagcagcagcagcagcagcagcagcagcagcagcagcagcngccgccgccgccgggccggccgccgccncctcntcatcatcngccgccgccgcnccagcagcngctgctcgtagncnngaaganga

SEQ ID NO: 2 - DNA, 267 bp, Artificial Sequence
  source: /note=“Description of artificial sequence Synthetic polynucleotide”
gtnnangntaccganggcctagaatgagtctaggaagacctgtaccgtttgcgatggccacaatgattgtcagcctgctctcgtcagcttggcgccttcnagctcccatcaagtccttccagcagcagcagcagcagcagcagcagcagcagcagcagcagcagcagcaacagccgccnccnccgccgccgccgccgccgccgccgccncctcctcntcntcntcngcngccgccgcagnannngcngctgctgctgcagnagacga

SEQ ID NO: 3 - DNA, 264 bp, Artificial Sequence
  source: /note=“Description of artificial sequence Synthetic polynucleotide”
gttgnnctnatnagncctagatgagtctaggaagagctgtaccgttgggatggccacaatgattctgaccctgctctcgtcagcttggcgcctgaaggccttcgagtccctcaagtccttccagcagcagcagcagcagcagcagcagcagcagcagcagcagcagcagcagcagcaacagccgccaccgccgccgccgccgccgcctcctcntcatcttcntcagccgccgcngnaggnacagcngctgctgccntcagncga

(19) United States
(12) Patent Application Publication
(10) Pub. No.: US 2010/0151470 A1
(43) Pub. Date: Jun. 17, 2010

(54) METHODS AND COMPOSITIONS FOR LOCATING SNP HETEROZYGOSITY FOR ALLELE SPECIFIC DIAGNOSIS AND THERAPY

(75) Inventors: PHILLIP D. ZAMORE, NORTHBORO, MA (US); NEIL ARONIN, NEWTONVILLE, MA (US)

Correspondence Address: LAHIVE & COCKFIELD, LLP;FLOOR 30, SUITE 3000, ONE POST OFFICE SQUARE, BOSTON, MA, 02109, US

(73) Assignee: UNIVERSITY OF MASSACHUSETTS, BOSTON, MA (US)

(21) Appl. No.: 12/609,474

(22) Filed: Oct. 30, 2009

Related U.S. Application Data
(63) Continuation of application No. PCT/US2008/005728, filed on May 1, 2008.
(60) Provisional application No. 60/927,018, filed on May 1, 2007.

(51) Int. Cl. C12Q 1/68

(52) U.S. Cl. 435/6

(57) ABSTRACT

The present invention provides methods for the rapid and cost effective identification of the presence of a disease-associated mutation and a particular SNP in the same allele of a gene without the need to clone and sequence the entire gene. The compositions and methods of the invention are useful for identification of patient to subpopulations amenable to treatment as part of a therapeutic strategy for treating genetic disorders, for example, dominant, gain-of-function gene mutations, for example, Huntington's Disease (HD).

### RELATED APPLICATIONS

This application claims the benefit of PCT/US2008/005728 filed on May 1, 2008, which claims the benefit of U.S. Provisional Patent Application No. 60/927,018, filed on May 1, 2007, the entire contents of which are hereby incorporated herein by reference.

### STATEMENT AS TO SPONSORED RESEARCH

This invention was made with government support under grant no. NS038194 awarded by the National Institutes of Health. The government has certain rights in the invention.

### BACKGROUND OF THE INVENTION

RNA interference (RNAi) is the mechanism of sequence-specific, post-transcriptional gene silencing initiated by double-stranded RNAs (dsRNA) homologous to the gene being suppressed. dsRNAs are processed by Dicer, a cellular ribonuclease III, to generate duplexes of about 21 nt with 3′-overhangs (small interfering RNA, siRNA) which mediate sequence-specific mRNA degradation. In mammalian cells siRNA molecules are capable of specifically silencing gene expression without induction of the unspecific interferon response pathway. RNA silencing agents have received particular interest as research tools and therapeutic agents for their ability to knock down expression of a particular protein with a high degree of sequence specificity.

Diseases caused by dominant, gain-of-function gene mutations develop in heterozygotes bearing one mutant and one wild type copy of the gene. One group of inherited gain-of-function disorders are known as the trinucleotide repeat diseases. The common genetic mutation among these diseases is an increase in a series of a particular trinucleotide repeat. To date, the most frequent trinucleotide repeat is CAG, which codes for the amino acid glutamine. At least 9 CAG repeat diseases are known and there are more than 20 varieties of these diseases, including Huntington's disease, Kennedy's disease and many spinocerebellar diseases. These disorders share a neurodegenerative component in the brain and/or spinal cord. Each disease has a specific pattern of neurodegeneration in the brain and most have an autosomal dominant inheritance. The onset of the diseases generally occurs at 30 to 40 years of age, but in Huntington's disease CAG repeats in the huntingtin gene of >60 portend a juvenile onset. Research has shown that the genetic mutation (increase in length of CAG repeats from normal <36 in the huntingtin gene to >36 in disease) is associated with the synthesis of a mutant huntingtin protein, which has >36 polyglutamines (Aronin et al., 1995). It has also been shown that the mutant protein forms cytoplasmic aggregates and nuclear inclusions (Difiglia et al., 1997) and associates with vesicles (Aronin et al., 1999). The exact mechanism whereby the mutant protein causes cell degeneration is not clear, but the origin of the cellular toxicity is known to be the mutant protein. Hence, the ability to silence expression of the mutant allele would effectively cure the disease.

The sequence specificity of RNA silencing agents is particularly useful for allele-specific silencing of dominant, gain-of-function gene mutations. However, in the case of Hungtinton's disease, although it would be highly desirable to silence expression of the mutant Huntington protein, RNAi methodologies targeting CAG repeats cannot be used without risking widespread destruction of normal CAG repeat-containing mRNAs. Thus instead of targeting the CAG repeats, single nucleotide polymorphisms (SNPs) specific to the disease-associated allele are made the targets of site-specific RNAi.

A major hurdle to using allele-specific SNP heterozygosities as RNAi targets is the identification the specific SNP nucleotides present on the disease-associated allele. The current approaches to this problem involve cloning and sequencing the patient's (or the patient's parents) entire disease-associated allele. In practical terms, such sequencing can be extremely costly and labor intensive, since it requires evaluating thousands of nucleotides (in the case of Huntington's disease). Thus, a rapid and cost-effective method for the identification of the specific SNP nucleotides associated with the disease-associated allele would be invaluable for the diagnosis of such a disease as well as subsequent treatment of using site-specific gene silencing.

### SUMMARY OF THE INVENTION

The present invention provides novel methods and compositions for identifying the presence of a disease-associated mutation and associated SNP in the same allele of a gene, without the need to clone and sequence the entire gene or even large portions thereof. The compositions and methods of the invention are also useful for the identification of patient subpopulations amenable to treatment as part of a therapeutic strategy for treating disorders having a genetic component. Genetic disorders particularly well-suited for identification and treatment, as disclosed herein, are those disorders caused or associated with dominant, gain-of-function gene mutations, for example, trinucleotide repeat gene mutations (e.g. Huntington's Disease (HD)). Other genetic disorders suitable for diagnosis and treatment according to the invention are those encoded by large alleles which are difficult to clone and sequence (e.g., a mutated dystrophin allele (2.5 megabases) which can cause Duchenne's muscular dystrophy).

Accordingly, the invention has several advantages which include, but are not to limited to, the following,
providing methods for identifying the presence of a disease-associated mutation and associated SNP nucleotide in the same allele of a gene, without the need to clone and sequence the entire gene,
providing methods of treating a subject having, or at risk for, a disease characterized, or caused by, the disease associated mutation by targeting the associated SNP with a gene silencing agent,
providing methods for identifying patients, amenable to SNP-targeted RNAi therapy, and
providing kits for detecting the presence of a disease-associated mutation and associated SNP nucleotide in the same nucleic acid molecule, suitable for use in diagnosis and/or SNP-targeted RNAi therapy.

Other features and advantages of the invention will be apparent from the following detailed description, and from the claims.

### BRIEF DESCRIPTION OF THE DRAWINGS

FIG. 1. Shows a schematic of the techniques disclosed herein.

FIG. 2. PCR amplification of htt exon 1 from postmortem brain samples from HD patients HD sample. cDNAs were produced by long-range reverse transcription using postmortem patient brain tissues. M: 100 by DNA ladder; A-G: patient samples with various numbers of CAG repeats.

FIG. 3. Amplification of cDNA spanning the exon 1 and SNP site of interests by long-range PCR. M: 1 kb DNA ladder, A: primers flank exon 1 and exon 25; B: primers flank exon 1 and exon 25 exon, C: primers flank exon 1 and exon exon 39, D: primers flank exon 1 and exon 50. Note that exon 1 contains the mutation that causes disease and exons 25, 29, 39, and 50 bear SNPs that are heterozygous.

FIG. 4. Circularization of Kas I digested cDNA. M: 1 kb DNA ladder. L: linear; C1: ligation reaction at 2.5 ng/ul of DNA, C2: 0.25 ng/ul; C3: 0.025 ng/ul.

FIG. 5. Inverse PCR products separated by agarose electrophoresis. M: 100 by DNA ladder, A-D: inverse PCR products of joint SNP at exon 25 and exon 1. Note that DNA with mutant exon 1 migrates slower than normal exon 1.

FIG. 6. Representative sequencing traces of purified inverse PCR products to containing joint core sections (SNP at exon 25 and CAG repeats in exon 1) of brain samples from HD patient. Note that because the patient sample examined is heterozygous for SNP at exon 25, each of the SNP is shown to connect with normal or mutant exon 1 alleles. 6-A: mutant allele, arrow shows adenine (A); 6-B: normal allele, arrow shows guanine (G).

FIG. 7. Representative sequencing trace of an inverse PCR product containing joint core sections (SNP at exon 25 and CAG repeats in exon 1) of fresh blood from an anonymous donor.

### DETAILED DESCRIPTION OF THE INVENTION

In order to provide a clear understanding of the specification and claims, the following definitions are conveniently provided below.

### DEFINITIONS

So that the invention may be more readily understood, certain terms are first defined.

As used herein, the term “RNA silencing” or “gene silencing” refers to a group of sequence-specific regulatory mechanisms (e.g. RNA interference (RNAi), transcriptional gene silencing (TGS), post-transcriptional gene silencing (PTGS), quelling, co-suppression, and translational repression) mediated by RNA molecules which result in the inhibition or “silencing” of the expression of a corresponding protein-coding gene. RNA silencing has been observed in many types of organisms, including plants, animals, and fungi.

The term “discriminatory RNA silencing” refers to the ability of an RNA molecule to substantially inhibit the expression of a “first” or “target” polynucleotide sequence while not substantially inhibiting the expression of a “second” or “non-target” polynucleotide sequence”, e.g., when both polynucleotide sequences are present in the same cell. In certain embodiments, the target polynucleotide sequence corresponds to a target gene, while the non-target polynucleotide sequence corresponds to a non-target gene. In other embodiments, the target polynucleotide sequence corresponds to a target allele, while the non-target polynucleotide sequence corresponds to a non-target allele. In certain embodiments, the target polynucleotide sequence is the DNA sequence encoding the regulatory region (e.g. promoter or enhancer elements) of a target gene. In other embodiments, the target polynucleotide sequence is a target mRNA encoded by a to target gene.

The term “target gene” is a gene whose expression is to be substantially inhibited or “silenced.” This silencing can be achieved by RNA silencing, e.g. by cleaving the mRNA of the target gene or translational repression of the target gene. The term “non-target gene” is a gene whose expression is not to be substantially silenced. In one embodiment, the polynucleotide sequences of the target and non-target gene (e.g. mRNA encoded by the target and non-target genes) can differ by one or more nucleotides. In another embodiment, the target and non-target genes can differ by one or more polymorphisms. In another embodiment, the target and non-target genes can share less than 100% sequence identity. In another embodiment, the non-target gene may be a homolog (e.g. an ortholog or paralog) of the target gene.

A “target allele” or “target gene” or “target SNP” is an allele, gene, or SNP whose expression is to be selectively inhibited or “silenced.” This silencing can be achieved by RNA silencing, e.g. by cleaving the mRNA of the target gene or target allele by a siRNA. The term “non-target allele” is a allele whose expression is not to be substantially silenced. In certain embodiments, the target and non-target alleles can correspond to the same target gene. In other embodiments, the target allele corresponds to a target gene, and the non-target allele corresponds to a non-target gene. In one embodiment, the polynucleotide sequences of the target and non-target alleles can differ by one or more nucleotides. In another embodiment, the target and non-target alleles can differ by one or more allelic polymorphisms. In another embodiment, the target and non-target alleles can share less than 100% sequence identity.

The term “polymorphism” as used herein, refers to a variation (e.g., one or more deletions, insertions, or substitutions) in a gene sequence that is identified or detected when the same gene sequence from different sources or subjects (but from the same organism) are compared. For example, a polymorphism can be identified when the same gene sequence from different subjects are compared. Identification of such polymorphisms is routine in the art, the methodologies being similar to those used to detect, for example, breast cancer point mutations. Identification can be made, for example, from DNA extracted from a subject's lymphocytes, followed by amplification of polymorphic regions using specific primers to said polymorphic region. Alternatively, the polymorphism can be identified when two alleles of the same gene are compared.

A variation in sequence between two alleles of the same gene within an organism is referred to herein as an “allelic polymorphism”. The polymorphism can be at a nucleotide within a coding region but, due to the degeneracy of the genetic code, no change in amino acid sequence is encoded. Alternatively, polymorphic sequences can encode a different amino acid at a particular position, but the change in the amino acid does not affect protein function. Polymorphic regions can also be found in non-encoding regions of the gene.

The term “gain-of-function mutation” as used herein, refers to any mutation in a gene in which the protein encoded by said gene (i.e., the mutant protein) acquires a function not normally associated with the protein (i.e., the wild type protein) causes or contributes to a disease or disorder. The gain-of-function mutation can be a deletion, addition, or substitution of a nucleotide or nucleotides in the gene which gives rise to the change in the function of the encoded protein. In one embodiment, the gain-of-function mutation changes the function of the mutant protein or causes interactions with other proteins. In another embodiment, the gain-of-function mutation causes a decrease in or removal of normal wild-type protein, for example, by interaction of the altered, mutant protein with said normal, wild-type protein.

As used herein, the term “gain-of-function disorder”, refers to a disorder characterized by a gain-of-function mutation. In one embodiment, the gain-of-function disorder is a neurodegenerative disease caused by a gain-of-function mutation, e.g., polyglutamine disorders and/or trinucleotide repeat diseases, for example, Huntington's disease. In another embodiment, the gain-of-function disorder is caused by a gain-of-function in an oncogene, the mutated gene product being a gain-of-function mutant, e.g., cancers caused by a mutation in the ret oncogene (e.g., ret-1), for example, endocrine tumors, medullary thyroid tumors, parathyroid hormone tumors, multiple endocrine neoplasia type2, and the like. Additional exemplary gain-of-function disorders include Alzheimer's, human immunodeficiency disorder (HIV), and slow channel congenital myasthenic syndrome (SCCMS).

The term “trinucleotide repeat diseases” as used herein, refers to any disease or disorder characterized by an expanded trinucleotide repeat region located within a gene, the expanded trinucleotide repeat region being causative of the disease or disorder. Examples of trinucleotide repeat diseases include, but are not limited to spino-cerebellar ataxia type 12 spino-cerebellar ataxia type 8, fragile X syndrome, fragile XE Mental to Retardation, Friedreich's ataxia and myotonic dystrophy. Preferred trinucleotide repeat diseases for treatment according to the present invention are those characterized or caused by an expanded trinucleotide repeat region at the 5′ end of the coding region of a gene, the gene encoding a mutant protein which causes or is causative of the disease or disorder. Certain trinucleotide diseases, for example, fragile X syndrome, where the mutation is not associated with a coding region may not be suitable for treatment according to the methodologies of the present invention, as there is no suitable mRNA to be targeted by RNAi. By contrast, disease such as Friedreich's ataxia may be suitable for treatment according to the methodologies of the invention because, although the causative mutation is not within a coding region (i.e., lies within an intron), the mutation may be within, for example, an mRNA precursor (e.g., a pre-spliced mRNA precursor).

The term “polyglutamine disorder” as used herein, refers to any disease or disorder characterized by an expanded of a (CAG) repeats at the 5′ end of the coding region (thus encoding an expanded polyglutamine region in the encoded protein). In one embodiment, polyglutamine disorders are characterized by a progressive degeneration of nerve cells. Examples of polyglutamine disorders include but are not limited to: Huntington's disease, spino-cerebellar ataxia type 1, spino-cerebellar ataxia type 2, spino-cerebellar ataxia type 3 (also know as Machado-Joseph disease), and spino-cerebellar ataxia type 6, spino-cerebellar ataxia type 7 and dentatoiubral-pallidoluysian atrophy.

The term “polyglutamine domain,” as used herein, refers to a segment or domain of a protein that consist of a consecutive glutamine residues linked to peptide bonds. In one embodiment the consecutive region includes at least 5 glutamine residues.

The term “expanded polyglutamine domain” or “expanded polyglutamine segment”, as used herein, refers to a segment or domain of a protein that includes at least 35 consecutive glutamine residues linked by peptide bonds. Such expanded segments are found in subjects afflicted with a polyglutamine disorder, as described herein, whether or not the subject has shown to manifest symptoms.

The term “trinucleotide repeat” or “trinucleotide repeat region” as used herein, refers to a segment of a nucleic acid sequence e.g.,) that consists of consecutive repeats of a particular trinucleotide sequence. In one embodiment, the trinucleotide repeat includes at least 5 consecutive trinucleotide sequences. Exemplary trinucleotide to sequences include, but are not limited to, CAG, CGG, GCC, GAA, CTG, and/or CGG.

The term “RNA silencing agent” refers to an RNA which is capable of inhibiting or “silencing” the expression of a target gene. In certain embodiments, the RNA silencing agent is capable of preventing complete processing (e.g, the full translation and/or expression) of a mRNA molecule through a post-transcriptional silencing mechanism. RNA silencing agents include small (<50 b.p.), noncoding RNA molecules, for example RNA duplexes comprising paired strands, as well as precursor RNAs from which such small non-coding RNAs can be generated. Exemplary RNA silencing agents include siRNAs, miRNAs, siRNA-like duplexes, and dual-function oligonucleotides as well as precursors thereof. In one embodiment, the RNA silencing agent is capable of inducing RNA interference. In another embodiment, the RNA silencing agent is capable of mediating translational repression.

The term “nucleoside” refers to a molecule having a purine or pyrimidine base covalently linked to a ribose or deoxyribose sugar. Exemplary nucleosides include adenosine, guanosine, cytidine, uridine and thymidine. Additional exemplary nucleosides include inosine, 1-methyl inosine, pseudouridine, 5,6-dihydrouridine, ribothymidine, ²N-methylguanosine and ^{2,2}N,N-dimethylguanosine (also referred to as “rare” nucleosides). The term “nucleotide” refers to a nucleoside having one or more phosphate groups joined in ester linkages to the sugar moiety. Exemplary nucleotides include nucleoside monophosphates, diphosphates and triphosphates. The terms “polynucleotide” and “nucleic acid molecule” are used interchangeably herein and refer to a polymer of nucleotides joined together by a phosphodiester linkage between 5′ and 3′ carbon atoms.

The term “RNA” or “RNA molecule” or “ribonucleic acid molecule” refers to a polymer of ribonucleotides. The term “DNA” or “DNA molecule” or deoxyribonucleic acid molecule” refers to a polymer of deoxyribonucleotides. DNA and RNA can be synthesized naturally (e.g., by DNA replication or transcription of DNA, respectively). RNA can be post-transcriptionally modified. DNA and RNA can also be chemically synthesized. DNA and RNA can be single-stranded (i.e., ssRNA and ssDNA, respectively) or multi-stranded (e.g., double stranded, i.e., dsRNA and dsDNA, respectively). “mRNA” or “messenger RNA” is single-stranded RNA that specifies the amino acid sequence of one or more polypeptide chains. This information is translated to during protein synthesis when ribosomes bind to the mRNA.

The term “RNA interference” (“RNAi”) refers to a selective intracellular degradation of RNA. RNAi occurs in cells naturally to remove foreign RNAs (e.g., viral RNAs). Natural RNAi proceeds via fragments cleaved from free dsRNA which direct the degradative mechanism to other similar RNA sequences. Alternatively, RNAi can be initiated by the hand of man, for example, to silence the expression of target genes.

The term “translational repression” refers to a selective inhibition of mRNA translation. Natural translational repression proceeds via miRNAs cleaved from shRNA precursors. Both RNAi and translational repression are mediated by RISC. Both RNAi and translational repression occur naturally or can be initiated by the hand of man, for example, to silence the expression of target genes.

An RNA silencing agent having a strand which is “sequence sufficiently complementary to a target mRNA sequence to direct target-specific RNA interference (RNAi)” means that the strand has a sequence sufficient to trigger the destruction of the target mRNA by the RNAi machinery or process.

The term “in vitro” has its art recognized meaning, e.g., involving purified reagents or extracts, e.g., cell extracts. The term “in vivo” also has its art recognized meaning, e.g., involving living cells, e.g., immortalized cells, primary cells, cell lines, and/or cells in an organism.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

Various aspects of the invention are described in further detail in the following subsections.

### 1. Overview

The present invention provides novel methods for identifying the presence of a disease-associated mutation and a particular SNP in the same allele of a gene without the need to clone and sequence the entire gene. The method of the invention is especially to suited to situations where the disease-associated mutation and heterozygous SNP are a large distance apart in the linear DNA sequence of the disease-associated allele (e.g., the huntingtin gene).

In one embodiment, mRNA, from a patient suffering from dominant gain-of-function disease, is isolated and converted, in vitro, into cDNA. A fragment of the cDNA is amplified using standard art recognized methods (e.g., PCR) using specific primers to generate a DNA fragment containing both the disease-associated mutation and a heterozygous SNP allele wherein, the disease-associated mutation and heterozygous SNP allele are in close proximity to the termini of the DNA fragment. The DNA fragment is then subject to intramolecular ligation to generate a circular DNA species wherein the disease-associated mutation and heterozygous SNP allele are in adjacent regions of the circular DNA species. A portion of the circular DNA species containing the disease-associated mutation and heterozygous SNP allele is then amplified using standard art recognized methods (e.g., PCR) and the amplified portion is subject to screening for the presence of said disease-associated mutation and heterozygous SNP allele using standard art recognized methods (e.g., DNA sequencing or hybridization).

In another embodiment, mRNA from a patient suffering from dominant gain-of-function disease, is isolated and subject to in vitro SNP-specific, discriminatory RNA silencing (e.g., RNAi-mediated cleavage) to generate 2 fragments. The RNA fragments are then subject to intramolecular ligation to generate a circular RNA species. A region of the circular RNA species containing the site of the disease-associated mutation and the ligation site is amplified using standard art recognized methods (e.g., RT-PCR) and the amplified region is subject to screening for the presence of said disease-associated mutation using standard art recognized methods (e.g., DNA sequencing or hybridization). Only the allele containing the specific SNP nucleotide will be cleaved, circularized and amplified. Hence, detection of the disease-associated mutation in the amplified region confirms linkage of the disease-associated mutation and the specific SNP nucleotide in the same allele.

In another aspect, the invention offers a method of treating a subject having or at risk for a disease arising from a disease-associated mutation identified according to the methods of the invention.

In another aspect, the invention offers a kit for identifying the presence of a to disease-associated mutation and a particular SNP in the same allele of a gene without the need to clone and sequence the entire gene.

In another aspect, the invention offers a method for identifying a patient or patient subpopulation amenable to discriminatory RNA silencing (e.g., SNP-targeted RNAi) therapy wherein the patient or patient subpopulation is first identified as in need of such therapy according to methods of the invention.

### 2. Selecting a Nucleic Acid Target

The present invention provides novel methods and compositions for identifying the presence of a disease-associated mutation and associated SNP in the same allele of a gene. In one embodiment the methods of the invention can also be used to identify the presence of any two or more nucleic acid sequence variants in a linear nucleic acid molecule. Exemplary target nucleic acids include, but are not limited to, RNA and DNA.

In certain exemplary aspects, the target mRNA molecule of the invention comprises a polymorphism or mutation but a sequence with a high degree of overall sequence identity (e.g. 80%, 90%, 92%, 95%, 98% or greater) with a second, non-target, mRNA that lacks the polymorphism or mutation. In certain embodiments, the target mRNA is encoded by the same gene that encodes the non-target mRNA. In other embodiments, the target mRNA is encoded by a different gene than that which encodes the non-target mRNA. In certain embodiments, the target mRNA has a high degree of sequence identity with a non-target mRNA that encodes a protein having a different function that the protein encoded by the target mRNA. In other embodiments, the target mRNA encodes a protein which performs the same biochemical function as the protein encoded by the non-target mRNA.

In preferred embodiments, the target mRNA comprises an allelic polymorphism or mutation that is specific to a particular allele of a gene and the non-target mRNA is encoded by a second allele (e.g. the wild-type allele) of the same gene. Accordingly, an object of the invention is to silence the expression of target mRNA which are associated with diseases or disorders (e.g. gain-of-function disorders), without substantially silencing the expression of a non-target (e.g., wild type mRNA.

2.1. Target Nucleic Acids Associated with Gain-of-Function Disorders

The term “gain-of-function mutation” as used herein, refers to any mutation in a gene in which the protein encoded by said gene (i.e., the mutant protein) acquires a function not normally associated with the protein (i.e., the wild type protein) causes or contributes to a disease or disorder. The gain-of-function mutation can be a deletion, addition, or substitution of a nucleotide or nucleotides in the gene which gives rise to the change in the function of the encoded protein.

In one embodiment, the gain-of-function mutation changes the function of the mutant protein or causes interactions with other proteins. In another embodiment, the gain-of-function mutation causes a decrease in or removal of normal wild-type protein, for example, by interaction of the altered, mutant protein with said normal, wild-type protein.

Gain-of-function mutations may give rise to gain-of-function diseases or disorders, including neurodegenerative disease. For example, Amyotrophic Lateral Sclerosis, Alzheimer's disease, Huntington's disease, and Parkinson's disease are associated with gain-of-function mutations in the genes encoding SOD1 (see Rosen et al., Nature, 362, 59-62, 1993; Rowland, Proc. Natl. Acad. Sci. USA, 92, 1251-1253, 1995), Amyloid Precursor Protein or APP (see Ikezu et al, EMBO J., (1996), 15(10):2468-75), Huntingtin or htt (see Rubinsztein, Trends Genet., (2002), 18(4):202-9), and alpha-synuclein (see, for example, Cuervo et al., Science, (2004), 305(5688): 1292-5), respectively. In another embodiment, disease or disorders of the present invention include neurodegenerative disease caused by a gain-of-function mutation in an oncogene, e.g., cancers caused by a mutation in the ret oncogene (e.g., ret-1), for example, gastrointestinal cancers, endocrine tumors, medullary thyroid tumors, parathyroid hormone tumors, multiple endocrine neoplasia type2, and the like.

The compositions of the invention are particularly well-suited for silencing the expression of gain-of-function disorders characterized by polymorphic regions (i.e., regions containing allele-specific or allelic polymorphisms, e.g. single-nucleotide polymorphisms (SNPs)) or point mutations (e.g. a point mutation occurring in a single allele in the mutant gene) where silencing the expression of the mutant allele, but not the wild type allele, is required. In a particularly preferred embodiment, the RNA silencing agents of the invention are capable of allelic discrimination with single nucleotide to specificity.

In another embodiment, a gain-of-function disorder of the present invention is a polyglutamine disorder. Polyglutamine disorders are a class of disease or disorders characterized by a common genetic mutation. In particular, the disease or disorders are characterized by an expanded repeat of the trinucleotide CAG which gives rise, in the encoded protein, to an expanded stretch of glutamine residues. Polyglutamine disorders are similar in that the diseases are characterized by a progressive degeneration of nerve cells.

Despite their similarities, polyglutamine disorders occur on different chromosomes and thus occur on entirely different segments of DNA. Examples of polyglutamine disorders include Huntington's disease, Dentatorubropallidoluysian Atrophy, Spinobulbar Muscular atrophy, Spinocerebellar Ataxia Type 1, Spinocerebellar Ataxia Type 2, Spinocerebellar Ataxia Type 3, Spinocerebellar Ataxia Type 6 and Spinocerebellar Ataxia Type 7 (Table 3). Polyglutamine disorders of the invention are characterized by (e.g., domains having between about 30 to 35 glutamine residues, between about 35 to 40 glutamine residues, between about 40 to 45 glutamine residues and having about 45 or more glutamine residues. The polyglutamine domain typically contains consecutive glutamine residues (Q n>36).

In one preferred embodiment, the disease or disorder of the present invention is Huntingtin's disease.

### 2.2. Huntington's Disease

In a preferred embodiment, the RNA silencing agents of the invention are designed to target polymorphisms (e.g. single nucleotide polymorphisms) in the mutant human huntingtin protein (htt) for the treatment of Huntington's disease.

Huntington's disease, inherited as an autosomal dominant disease, causes impaired cognition and motor disease. Patients can live more than a decade with severe debilitation, before premature death from starvation or infection. The disease begins in the fourth or fifth decade for most cases, but a subset of patients manifest disease in teenage years.

The genetic mutation for Huntington's disease is a lengthened CAG repeat in the to huntingtin gene. CAG repeat varies in number from 8 to 35 in normal individuals (Kremer et al., 1994). The genetic mutation e.g.,) an increase in length of the CAG repeats from normal less than 36 in the huntingtin gene to greater than 36 in the disease is associated with the synthesis of a mutant huntingtin protein, which has greater than 36 polyglutamates (Aronin et al., 1995).

In general, individuals with 36 or more CAG repeats will get Huntington's disease. Prototypic for as many as twenty other diseases with a lengthened CAG as the underlying mutation, Huntington's disease still has no effective therapy. A variety of interventions such as interruption of apoptotic pathways, addition of reagents to boost mitochondrial efficiency, and blockade of NMDA receptors—have shown promise in cell cultures and mouse model of Huntington's disease. However, at best these approaches reveal a short prolongation of cell or animal survival.

Huntington's disease complies with the central dogma of genetics: a mutant gene serves as a template for production of a mutant mRNA; the mutant mRNA then directs synthesis of a mutant protein (Aronin et al., 1995; DiFiglia et al., 1997). Mutant huntingtin (protein) probably accumulates in selective neurons in the striatum and cortex, disrupts as yet determined cellular activities, and causes neuronal dysfunction and death (Aronin et al., 1999; Laforet et al., 2001).

Because a single copy of a mutant gene suffices to cause Huntington's disease, the most parsimonious treatment would render the mutant gene ineffective. Theoretical approaches might include stopping gene transcription of mutant huntingtin, destroying mutant mRNA, and blocking translation. Each has the same outcome—loss of mutant huntingtin.

The disease gene linked to Huntington's disease is termed Huntington or (htt). The huntingtin locus is large, spanning 180 kb and consisting of 67 exons. The huntingtin gene is widely expressed and is required for normal development. It is expressed as 2 alternatively polyadenylated forms displaying different relative abundance in various fetal and adult tissues. The larger transcript is approximately 13.7 kb and is expressed predominantly in adult and fetal brain whereas the smaller transcript of approximately 10.3 kb is more widely expressed. The two transcripts differ with respect to their 3′ untranslated regions (Lin et al., 1993). Both messages are predicted to encode a 348 kilodalton protein containing 3144 amino acids. The genetic defect leading to Huntington's disease is believed to confer a new property on the mRNA or alter the function of the protein.

Exemplary single nucleotide polymorphisms (SNPs) in the huntingtin gene sequence can be found at positions 2886, 4034, 6912, 7222, and 7246 of the human htt gene. Additional single nucleotide polymorphisms in the huntingtin gene sequence are set forth in Table 1 below. Yet other exemplary SNPs are described in International Publication No. WO 2008/005562, filed Jul. 9, 2007, which is herein incorporated by reference in its entirety. In certain preferred embodiments, the SNP is a heterozygous SNP allele having an allelic frequency of at least 10% (e.g., at least 15%, 20%, 25%, 30%, 35%, 40% or more) in a sample population. In certain embodiments, the heterozygous SNP allele is found at a SNP site selected from the group consisting of RS362331, RS4690077, RS363125, 47 by into Exon 25, RS363075, RS362268, RS362267, RS362307, RS362306, RS362305, RS362304, and RS362303. In one embodiment, the SNP allele is present at SNP target site RS363125. In a particular embodiment, the SNP allele is a C nucleotide. In another particular embodiment, the SNP allele is a U nucleotide. In another embodiment, the SNP allele is present at SNP target site RS362331. In a particular embodiment, the SNP allele is an A nucleotide. In another particular embodiment, the SNP allele is a C nucleotide. In another embodiment, the SNP allele is present at position 171, e.g., an A171C polymorphism, in the huntingtin gene according to the sequence numbering in GenBank Accession No. NM_{—}002111 (Aug. 8, 2005).

In certain embodiments, RNA silencing agents of the invention may be designed according to the above exemplary teachings to target any of the single nucleotide polymorphisms described supra. Said RNA silencing agents comprise an antisense strand which is fully complementary with the single nucleotide polymorphism. In certain embodiments, the RNA silencing agent is a siRNA.

To validate the effectiveness by which siRNAs destroy mutant mRNAs (e.g., mutant huntingtin mRNA), the siRNA is incubated with mutant cDNA (e.g., mutant huntingtin cDNA) in a Drosophila-based in vitro mRNA expression system. Radiolabeled with ³²P, newly synthesized mutant mRNAs (e.g., mutant huntingtin mRNA) are detected autoradiographically on an agarose gel. The presence of cleaved mutant mRNA indicates mRNA nuclease activity. Suitable controls include omission of siRNA and use of wild-type huntingtin cDNA.

Alternatively, control siRNAs are selected having the same nucleotide composition as the selected siRNA, but without significant sequence complementarity to the appropriate target gene. Such negative controls can be designed by randomly scrambling the nucleotide sequence of the selected siRNA; a homology search can be performed to ensure that the negative control lacks homology to any other gene in the appropriate genome. In addition, negative control siRNAs can be designed by introducing one or more base mismatches into the sequence.

Sites of siRNA-mRNA complementation are selected which result in optimal mRNA specificity and maximal mRNA cleavage.

TABLE 1
Exemplary SNPs in the Huntingtin Gene.

| | Consensus | polymorphism | | db_xref |
|---|---|---|---|---|
| complement 103 | G | A | P6 | dbSNP: 396875 |
| complement 432 | T | C | P7 | dbSNP: 473915 |
| complement 474 | C | A | P8 | dbSNP: 603765 |
| 1509 | T | C | P9 | dbSNP: 1065745 |
| complement 1857 | T | C | P10 | dbSNP: 2301367 |
| 3565 | G | C, A | P11, P12 | dbSNP: 1065746 |
| 3594 | T | G | P13 | dbSNP: 1143646 |
| 3665 | G | C | P14 | dbSNP: 1065747 |
| complement 4122 | G | A | P15 | dbSNP: 363099 |
| complement 4985 | G | A | P16 | dbSNP: 363129 |
| complement 5480 | T | G | P17 | dbSNP: 363125 |
| 6658 | T | G | P18 | dbSNP: 1143648 |
| complement 6912 | T | C | P19 | dbSNP: 362336 |
| complement 7753 | G | A | P20 | dbSNP: 3025816 |
| complement 7849 | G | C | P21 | dbSNP: 3025814 |
| complement 8478 | T | C | P22 | dbSNP: 2276881 |
| 8574 | T | C | P23 | dbSNP: 2229985 |
| complement 9154 | C | A | P24 | dbSNP: 3025807 |
| 9498 | T | C | P25 | dbSNP: 2229987 |
| complement 9699 | G | A | P26 | dbSNP: 362308 |
| complement 9809 | G | A | P27 | dbSNP: 362307 |
| complement 10064 | T | C | P28 | dbSNP: 362306 |
| complement 10112 | G | C | P29 | dbSNP: 362268 |
| complement 10124 | G | C | P30 | dbSNP: 362305 |
| complement 10236 | T | G | P31 | dbSNP: 362304 |
| complement 10271 | G | A | P32 | dbSNP: 362303 |
| complement 10879 | G | A | P33 | dbSNP: 1557210 |
| complement 10883 | G | A | P34 | dbSNP: 362302 |
| complement 10971 | C | A | P35 | dbSNP: 3025805 |
| complement 11181 | G | A | P36 | dbSNP: 362267 |
| complement 11400 | C | A | P37 | dbSNP: 362301 |
| 11756 . . . 11757 | G | — | P38 | dbSNP: 5855774 |
| 12658 | G | A | P39 | dbSNP: 2237008 |
| complement 12911 | T | C | P40 | dbSNP: 362300 |
| complement 13040 | G | A | P41 | dbSNP: 2530595 |
| 13482 | G | A | P42 | dbSNP: 1803770 |
| 13563 | G | A | P43 | dbSNP: 1803771 |

While the instant invention primarily features targeting polymorphic regions in the target mutant gene (e.g., in mutant htt) distinct from the expanded CAG region mutation, the skilled artisan will appreciate that targeting the mutant region may have applicability as a therapeutic strategy in certain situations. Targeting the mutant region can be accomplished using siRNA that complements CAG in series. The siRNA^{cag} would bind to mRNAs with CAG complementation, but might be expected to have greater opportunity to bind to an extended CAG series. Multiple siRNA^{cag} would bind to the mutant huntingtin mRNA (as opposed to fewer for the wild type huntingtin mRNA); thus, the mutant huntingtin mRNA is more likely to be cleaved. Successful mRNA inactivation using this approach would also eliminate normal or wild-type huntingtin mRNA. Also inactivated, at least to some extent, could be other normal genes (approximately 70) which also have CAG repeats, where their mRNAs could interact to with the siRNA. This approach would thus rely on an attrition strategy—more of the mutant huntingtin mRNA would be destroyed than wild type huntingtin mRNA or the other approximately 69 mRNAs that code for polyglutamines

### 3. RNA Silencing Agents

The present invention features improved RNA silencing agents (e.g., siRNA and shRNAs) for conducting therapy upon diagnosis (e.g., according to the methods of the invention) of a disease-associated mutation and its linkage with a SNP that can be effectively targeted. Typically, the target sequence is an allelic polymorphism or point mutation (e.g., SNP as disclosed herein) which is unique to a mutant allele for which silencing is desired. Typically a siRNA molecule is used but other gene silencing agents can be substituted as appropriate.

An siRNA molecule of the invention is a duplex consisting of a sense strand and complementary antisense strand, the antisense strand having sufficient complementary to a target mRNA to mediate RNAi, in particular, and SNP associated with (having strong linkage with) a disease associated mutation as disclosed herein.

Preferably, the siRNA molecule has a length from about 10-50 or more nucleotides, i.e., each strand comprises 10-50 nucleotides (or nucleotide analogs). More preferably, the siRNA molecule has a length from about 16-30, e.g., 16, 17, 18, 19, 20, 21, 22, 23, 24, 25, 26, 27, 28, 29, or 30 nucleotides in each strand, wherein one of the strands is sufficiently complementary to a target region.

Preferably, the strands are aligned such that there are at least 1, 2, or 3 bases at the end of the strands which do not align (i.e., for which no complementary bases occur in the opposing strand) such that an overhang of 1, 2 or 3 residues occurs at one or both ends of the duplex when strands are annealed. Preferably, the siRNA molecule has a length from about 10-50 or more nucleotides, i.e., each strand comprises 10-50 nucleotides (or nucleotide analogs).

More preferably, the siRNA molecule has a length from about 16-30, e.g., 16, 17, 18, 19, 20, 21, 22, 23, 24, 25, 26, 27, 28, 29, or 30 nucleotides in each strand, wherein one of the strands is substantially complementary to a target region e.g., a gain-of-function gene target region, and the other strand is identical or substantially identical to the first strand.

Sequence identity may be determined by sequence comparison and alignment to algorithms known in the art. To determine the percent identity of two nucleic acid sequences (or of two amino acid sequences), the sequences are aligned for optimal comparison purposes (e.g., gaps can be introduced in the first sequence or second sequence for optimal alignment). The nucleotides (or amino acid residues) at corresponding nucleotide (or amino acid) positions are then compared. When a position in the first sequence is occupied by the same residue as the corresponding position in the second sequence, then the molecules are identical at that position. The percent identity between the two sequences is a function of the number of identical positions shared by the sequences (i.e., % homology=# of identical positions/total # of positions×100), optionally penalizing the score for the number of gaps introduced and/or length of gaps introduced.

The comparison of sequences and determination of percent identity between two sequences can be accomplished using a mathematical algorithm. In one embodiment, the alignment generated over a certain portion of the sequence aligned having sufficient identity but not over portions having low degree of identity (i.e., a local alignment). A preferred, non-limiting example of a local alignment algorithm utilized for the comparison of sequences is the algorithm of Karlin and Altschul (1990) Proc. Natl. Acad. Sci. USA 87:2264-68, modified as in Karlin and Altschul (1993) Proc. Natl. Acad. Sci. USA 90:5873-77. Such an algorithm is incorporated into the BLAST programs (version 2.0) of Altschul, et al. (1990) J. Mol. Biol. 215:403-10.

### 4. Methods of Introducing Nucleic Acids, Vectors, and Host Cells

RNA silencing agents of the invention may be directly introduced into the cell (i.e., intracellularly); or introduced extracellularly into a cavity, interstitial space, into the circulation of an organism, introduced orally, or may be introduced by bathing a cell or organism in a solution containing the nucleic acid. Vascular or extravascular circulation, the blood or lymph system, and the cerebrospinal fluid are sites where the nucleic acid may be introduced.

The RNA silencing agents of the invention can be introduced using nucleic acid delivery methods known in art including injection of a solution containing the nucleic acid, bombardment by particles covered by the nucleic acid, soaking the cell or organism in a solution of the nucleic acid, or electroporation of cell membranes in the presence of the nucleic acid. Other methods known in the art for introducing nucleic acids to cells to may be used, such as lipid-mediated carrier transport, chemical-mediated transport, and cationic liposome transfection such as calcium phosphate, and the like. The nucleic acid may be introduced along with other components that perform one or more of the following activities: enhance nucleic acid uptake by the cell or other-wise increase inhibition of the target gene.

The cell having the target gene may be from the germ line or somatic, totipotent or pluripotent, dividing or non-dividing, parenchyma or epithelium, immortalized or transformed, or the like. The cell may be a stem cell or a differentiated cell. Cell types that are differentiated include adipocytes, fibroblasts, myocytes, cardiomyocytes, endothelium, neurons, glia, blood cells, megakaryocytes, lymphocytes, macrophages, neutrophils, eosinophils, basophils, mast cells, leukocytes, granulocytes, keratinocytes, chondrocytes, osteoblasts, osteoclasts, hepatocytes, and cells of the endocrine or exocrine glands.

Depending on the particular target gene and the dose of RNA silencing agent material delivered, this process may provide partial or complete loss of function for the target gene. A reduction or loss of gene expression in at least 50%, 60%, 70%, 80%, 90%, 95% or 99% or more of targeted cells is exemplary Inhibition of gene expression refers to the absence (or observable decrease) in the level of protein and/or mRNA product from a target gene. Specificity refers to the ability to inhibit the target gene without manifest effects on other genes of the cell. The consequences of inhibition can be confirmed by examination of the outward properties of the cell or organism (as presented below in the examples) or by biochemical techniques such as RNA solution hybridization, nuclease protection, Northern hybridization, reverse transcription, gene expression monitoring with a microarray, antibody binding, enzyme linked immunosorbent assay (ELISA), Western blotting, radioimmunoassay (RIA), other immunoassays, and fluorescence activated cell analysis (FACS).

For RNA-mediated inhibition in a cell line or whole organism, gene expression is conveniently assayed by use of a reporter or drug resistance gene whose protein product is easily assayed. Such reporter genes include acetohydroxyacid synthase (AHAS), alkaline phosphatase (AP), beta galactosidase (LacZ), beta glucoronidase (GUS), chloramphenicol acetyltransferase (CAT), green fluorescent protein (GFP), horseradish peroxidase (HRP), luciferase (Luc), nopaline synthase (NOS), octopine synthase (OCS), and derivatives thereof. Multiple selectable markers are available that confer resistance to to ampicillin, bleomycin, chloramphenicol, gentamycin, hygromycin, kanamycin, lincomycin, methotrexate, phosphinothricin, puromycin, and tetracyclin. Depending on the assay, quantitation of the amount of gene expression allows one to determine a degree of inhibition which is greater than 10%, 33%, 50%, 90%, 95% or 99% as compared to a cell not treated according to the present invention. Lower doses of injected material and longer times after administration of RNA silencing agent may result in inhibition in a smaller fraction of cells (e.g., at least 10%, 20%, 50%, 75%, 90%, or 95% of targeted cells). Quantitation of gene expression in a cell may show similar amounts of inhibition at the level of accumulation of target mRNA or translation of target protein. As an example, the efficiency of inhibition may be determined by assessing the amount of gene product in the cell; mRNA may be detected with a hybridization probe having a nucleotide sequence outside the region used for the inhibitory double-stranded RNA, or translated polypeptide may be detected with an antibody raised against the polypeptide sequence of that region.

The RNA silencing agent may be introduced in an amount which allows delivery of at least one copy per cell. Higher doses (e.g., at least 5, 10, 100, 500 or 1000 copies per cell) of material may yield more effective inhibition; lower doses may also be useful for specific applications.

### 5. Methods of Treatment

The present invention provides for both prophylactic and therapeutic methods of treating a subject at risk of (or susceptible to) a disorder caused by a genetic disease, for example, a gain-of-function mutation (e.g., HD). In one embodiment, the invention provides an RNA silencing agent (e.g., RNAi agent) for suppressing the expression of the undesired gene product. It is understood that “treatment” or “treating” as used herein, is defined as the application or administration of a therapeutic agent (e.g., a RNAi agent or vector or transgene encoding same) to a patient, or application or administration of a therapeutic agent to an isolated tissue or cell line from a patient, who has a disease or disorder, a symptom of disease or disorder or a predisposition toward a disease or disorder, with the purpose to cure, heal, alleviate, relieve, alter, remedy, ameliorate, improve or affect the disease or disorder, the symptoms of the disease or disorder, or the predisposition toward disease.

### 6. Prophylactic Methods

In another aspect, the invention provides a method for preventing in a subject, a disease or condition associated with an aberrant or unwanted target gene expression or activity, by administering to the subject a therapeutic agent (e.g., a RNAi agent or vector or transgene encoding same). Subjects at risk for a disease which is caused or contributed to by aberrant or unwanted target gene expression or activity can be identified by, for example, any or a combination of diagnostic or prognostic assays as described herein. Administration of a prophylactic agent can occur prior to the manifestation of symptoms characteristic of the target gene aberrancy, such that a disease or disorder is prevented or, alternatively, delayed in its progression. Depending on the type of target gene aberrancy, for example, a target gene, target gene agonist or target gene antagonist agent can be used for treating the subject. The appropriate agent can be determined based on screening assays described herein.

### 7. Therapeutic Methods

In another aspect, the invention provides methods of modulating target gene expression, protein expression or activity for therapeutic purposes. Accordingly, in an exemplary embodiment, the modulatory method of the invention involves contacting a cell capable of expressing target gene with a therapeutic agent (e.g., RNAi agent or vector or transgene encoding same) that is specific for the target gene, in particular, target gene SNP region (e.g., is specific for the mRNA encoded by said gene or specifying the amino acid sequence of said protein) such that expression or one or more of the activities of target protein is modulated. These modulatory methods can be performed in vitro (e.g., by culturing the cell with the agent), in vivo (e.g., by administering the agent to a subject), or ex vivo. As such, the present invention provides methods of treating an individual afflicted with a disease or disorder characterized by aberrant or unwanted expression or activity of a target gene polypeptide or nucleic acid molecule Inhibition of target gene activity is desirable in situations in which target gene is abnormally unregulated and/or in which decreased target gene activity is likely to have a beneficial effect, for example, in achieving therapy for a gain-of-function disease.

### 8. Pharmacogenomics

In another aspect, the invention provides methods and compositions for to performing pharmacogenomics. The therapeutic agents (e.g., a RNAi agent or vector or transgene encoding same) of the invention can be administered to individuals to treat (prophylactically or therapeutically) disorders associated with aberrant or unwanted target gene activity (and targetable SNP). In conjunction with such treatment, pharmacogenomics (i.e., the study of the relationship between an individual's genotype and that individual's response to a foreign compound or drug) may be considered. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer a therapeutic agent as well as tailoring the dosage and/or therapeutic regimen of treatment with a therapeutic agent.

Pharmacogenomics deals with clinically significant hereditary variations in the response to drugs due to altered drug disposition and abnormal action in affected persons. See, for example, Eichelbaum, M. et al. (1996) Clin. Exp. Pharmacol. Physiol. 23(10-11): 983-985 and Linder, M. W. et al. (1997) Clin. Chem. 43(2):254-266

In one aspect, the methods of the invention provide information regarding the linkage of SNP nucleotides to disease-associated mutations in the same allele. In one embodiment, this information is used to select patients or patient subpopulations for treatment with SNP-specific RNAi-based therapies. In another embodiment this information is used to select patients or patient subpopulations for treatment with conventional FDA-approved therapies e.g., antibody, small molecule or peptide therapies.

### 9. Pharmaceutical Compositions

The invention pertains to uses of the above-described agents for therapeutic treatments as described herein. Accordingly, the modulators of the present invention can be incorporated into pharmaceutical compositions suitable for administration. Such compositions typically comprise an RNAi agent, e.g., an siRNA agent for carrying out gene silencing, and, optionally, a protein, antibody, or modulatory compound, if appropriate, and a pharmaceutically acceptable carrier. As used herein the language “pharmaceutically acceptable carrier” is intended to include any and all solvents, to dispersion media, coatings, antibacterial and antifungal agents, isotonic and absorption delaying agents, and the like, compatible with pharmaceutical administration.

The use of such media and agents for pharmaceutically active substances is well known in the art. Except insofar as any conventional media or agent is incompatible with the active compound, use thereof in the compositions is contemplated. Supplementary active compounds can also be incorporated into the compositions.

### 10. Other Applications of the Technology of the Invention

In another embodiment, the invention provides SNP sequence information for making diagnostic kits, or chips.

In another embodiment, the SNP sequence information or methodology disclosed herein can be used for forensic applications.

In another embodiment, the methods and compositions disclosed herein can be used for research purposes, for example genetic research on the distribution or migration of human populations.

In still another embodiment, the invention provides business methods for commercializing SNPs suitable for use in, for example, the making of diagnostic chips, kits, and pharmaceuticals for targeting disease associated mutations.

### EXEMPLIFICATION

Throughout the examples, the following materials and methods were used unless otherwise stated.

### Materials and Methods

The present invention employs many conventional molecular biology, microbiology and recombinant DNA techniques. Such techniques are explained fully in the literature. See for example, Sambrook et al., (1989) Molecular Cloning—A Laboratory Manual, Cold Spring Harbor Press, Sambrook and Russell, Molecular Cloning, Third Edition, Cold Spring Harbor Press (2000); Glover, (1985) DNA Cloning: A Practical Approach; Gait, (1984) Oligonucleotide Synthesis; Harlow & Lane, (1988) Antibodies-A Laboratory Manual, Cold Spring Harbor Press; Roe et al., (1996) DNA Isolation and Sequencing: Essential Techniques, John Wiley; and Ausubel et. al., (1995) Current Protocols in Molecular Biology, (1993) including supplements through May 2005, John Wiley & Sons.

In certain embodiments the present invention uses SNP-specific, in vitro RNAi to identify the presence of disease-associated mutations and specific SNP nucleotides in the same RNA molecule. The use of in vitro RNAi reactions is described in the art (Zamore et al., Cell, (2000), 101: 25-33; Haley et al., Methods, (2003), 30: 330-336; Tuschl et al., Genes Dev., (1999), 13:3191-3197).

In certain embodiments the present invention mRNA fragment derived from the cleavage of mRNA by a RISC complex in vitro are circularized. For 5′ mRNA fragments containing a 5′ cap, a preferred method is to treat with Tobacco Acid Pyrophosphatase (to remove the 5′ CAP from the mRNA) followed by ligation with an RNA ligase. 3′ mRNA fragments can be directly ligated with an RNA ligase.

In certain embodiments portions of DNA or RNA are amplified by PCR or RT-PCR. Specific oligonucleotide primers, complementary to the specific template, are synthesized by art recognized methods. Other techniques for carrying out the invention are disclosed in U.S. Ser. No. 11/022,055; PCT/US2004/029968; and 60/819,704.

### Example 1

### A Method for Determining the Presence of a Specific SNP Nucleotide in the Disease-Associated Allele in HD

The following example describes a novel method for determining the presence of a specific SNP nucleotide in the disease-associated allele in HD.

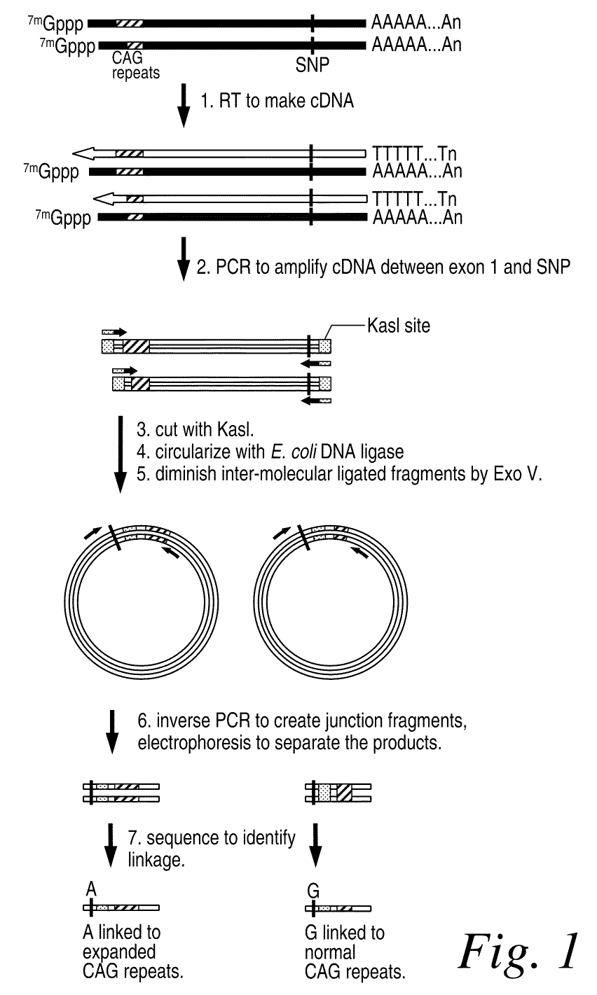

The method is illustrated in FIG. 1. Briefly, the full-length cDNA complementary to htt mRNA was generated from a patient with HD by reverse transcription. A portion of the cDNA was amplified by PCR using primers that flank exon 1 (which contains the expanded CAG repeat is HD) and the SNP of interest that is to heterozygous. Note that both primers are designed to bear a Kas I restriction sequence in their 5′ region. The resultant PCR product was digested with the Kas I restriction endonuclease and intramolecular religation performed using T4 DNA ligase to form a circular DNA species such that the SNP site and exon 1 are now adjacent to one another. A fragment of the circular DNA species containing the SNP site and exon 1 was amplified by inverse PCR. The mutant allele has an expanded CAG repeat region thus, the PCR products with mutant exon 1 migrate slower than those with wild-type exon 1 and can be separated by agarose gel electrophoresis. The two species of PCR products are isolated and purified separately from the agarose gel using standard art recognized methods and subject to DNA sequencing.

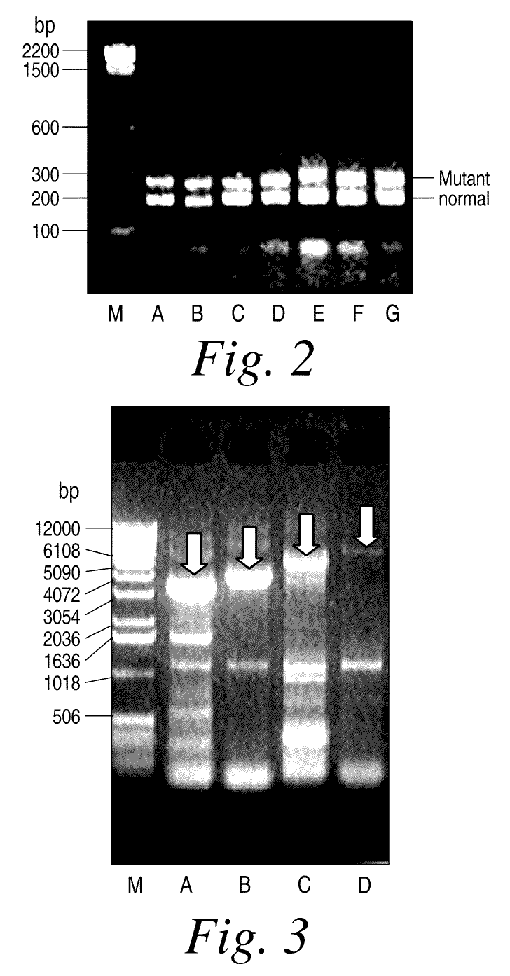

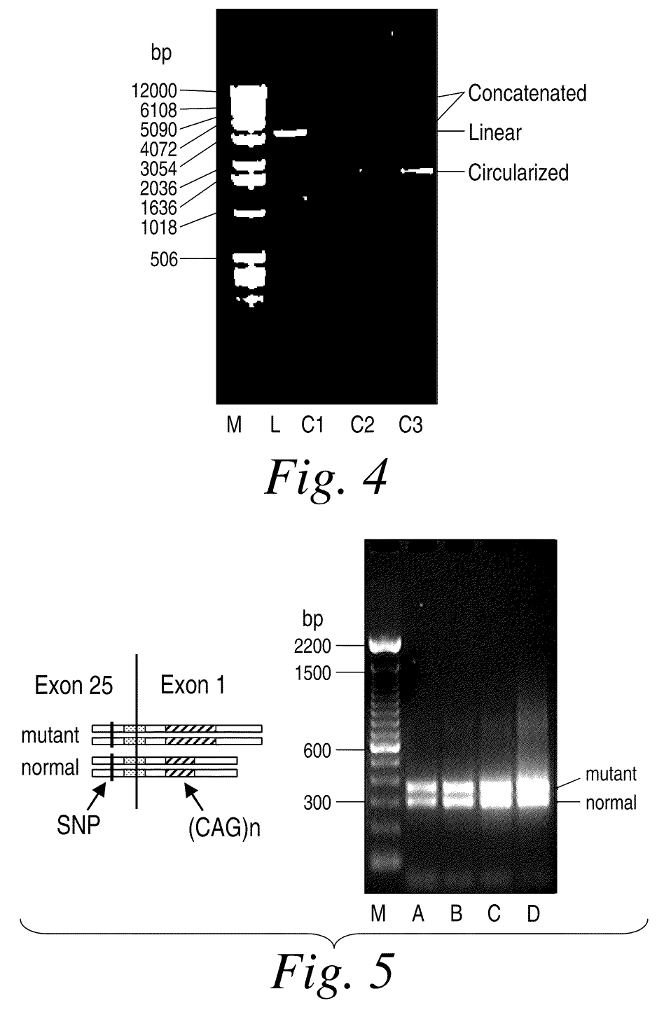

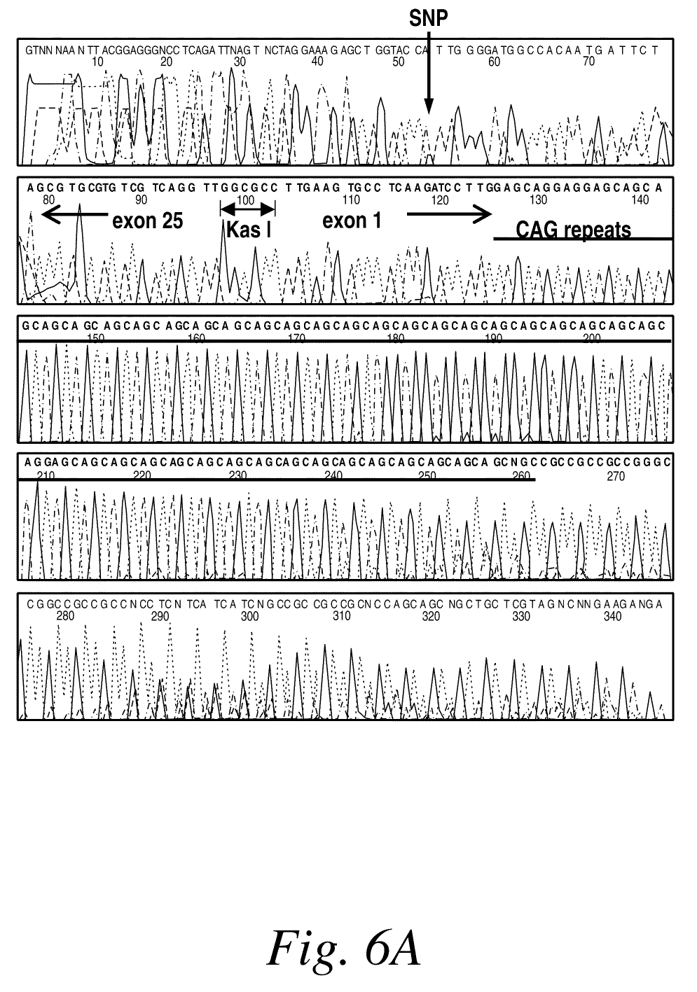

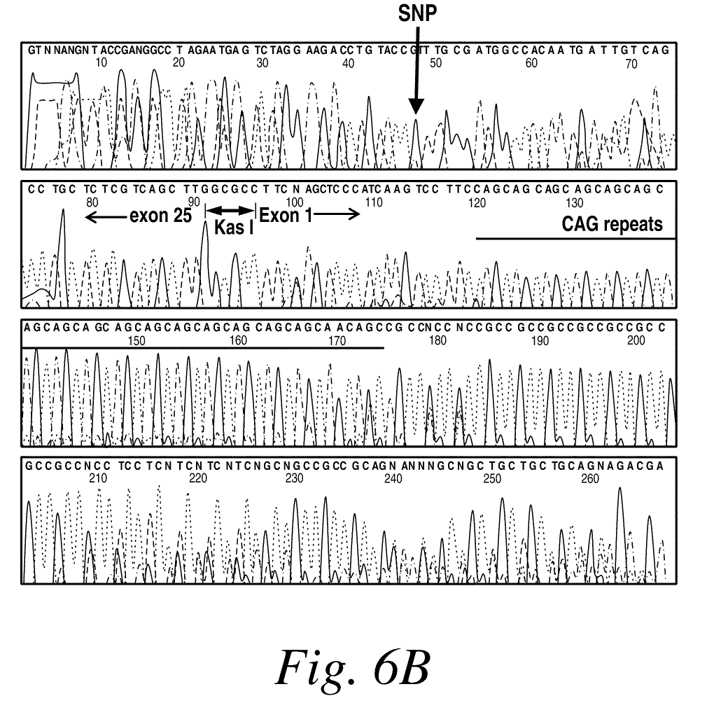

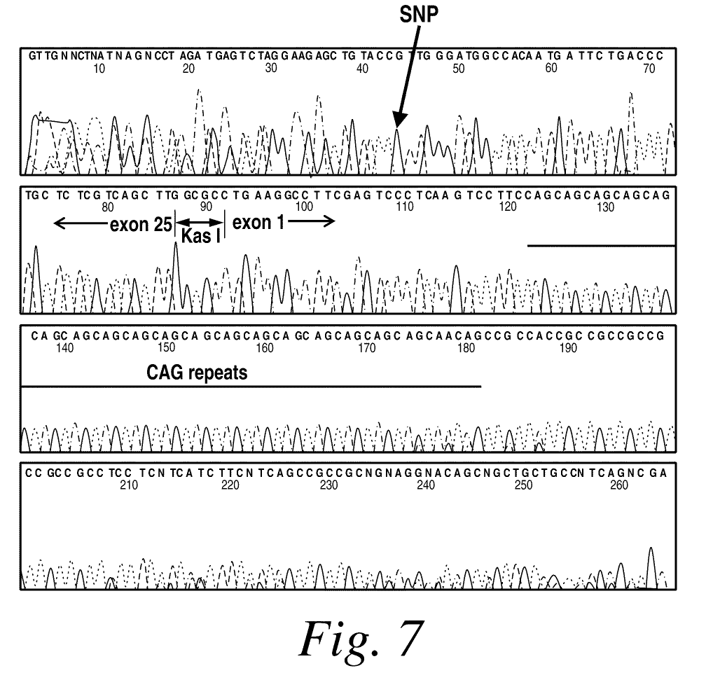

Accordingly, FIGS. 1-7 illustrate that the technique works exactly as described above. Specifically the sequence information presented in FIG. 6 conclusively demonstrates the linkage of a particular SNP to the expanded CAG region of the mutant htt allele. Hence the present invention provides a rapid, cost-effective and robust method for determining the linkage of a specific SNP nucleotide to the disease associated allele in HD. Moreover, the method can be used for determining the presence of any known SNP nucleotide in the disease-associated allele in HD by using appropriate oligonucleotide primer during the amplification steps. Further, the technique can be used for determining the linkage of any two or more known nucleotide variants in a disease associated allele. Further still, the technique can be used for determining the linkage of any two or more known nucleotide variants in a nucleic acid sequence.

### Example 2

### Analysis of Allele Specific SNP Heterozygosities in HD Patient Blood Samples

The following example illustrates the successful identification of allele specific SNP heterozygosities from HD patient blood samples using the methods of the invention.

Total RNA extracted from HD patient peripheral blood lymphocytes was used to synthesize full-length Htt cDNA. Long range PCR was then employed to amplify the to DNA region spanning from exon 1 (which contains the CAG repeats) to the heterozygous SNPs, which lie 1000's of base pairs away. The resultant PCR products were circularized by intramolecular ligation resulting in the juxtaposition of the CAG repeats and site of the SNP to be interrogated (see FIG. 1). A second PCR reaction using primers flanking exon 1 and the SNP site generated a small DNA fragment containing the exonl CAG repeats fused to the SNP site. The small size of this product, relative to the length of the CAG repeat allowed the PCR products from each allele to be readily separated by electrophoresis. Direct sequencing each PCR product established which nucleotide variant of the SNP was linked to the expanded and normal CAG repeats. Using this method, we have successfully identified the linkage between the disease-causing CAG expansion and 8 SNP sites in 17 HD patients (Table 2); these SNP sites were located 3300 to 11000 base pairs distal to the CAG repeats. Thus, the methods of the invention will be clinically useful for the selection of patient-specific siRNAs targeting only the mutant huntingtin allele.

TABLE 2
Analysis of Allele Specific SNP Heterozygosities in HD Patient Blood
Samples.

| Patient | | SNP | SNP |
|---|---|---|---|
| sample No. | SNP position | heterozygosity | Linkage |
| 2 | exon25 | G/A | M-G; N-A |
| 3 | exon29 | C/T | M-T; N-C |
| 10 | exon29 | C/T | M-C; N-T |
| 12 | exon29 | C/T | M-T; N-C |
| 3 | exon48 | A/G | M-G; N-A |
| 8 | exon48 | A/G | M-G; N-A |
| 10 | exon48 | A/G | M-G; N-A |
| 12 | exon48 | A/G | M-A; N-G |
| 3 | exon50 | T/C | M-T; N-C |
| 4 | exon50 | T/C | M-T; N-C |
| 10 | exon50 | T/C | M-T; N-C |
| 11 | exon50 | T/C | M-T; N-C |
| 3 | exon57 | A/G | M-A; N-G |
| 10 | exon57 | A/G | M-A; N-G |
| 12 | exon57 | A/A | M-G; N-A |
| 4 | exon61 | G/A | N-A; M-G |
| 5 | exon61 | G/A | N-G; M-A |
| 7 | exon61 | G/A | N-A; M-G |
| 9 | exon61 | G/A | N-A; M-G |
| 4 | 3′UTR (POS. 9633) | C/T | M-T; N-C |
| 5 | 3′UTR (POS. 9633) | C/T | M-T; N-C |
| 7 | 3′UTR (POS. 9633) | C/T | M-T; N-C |
| 8 | 3′UTR (POS. 9633) | C/T | M-T; N-C |
| 9 | 3′UTR (POS. 9633) | C/T | M-T; N-C |
| 11 | 3′UTR (POS. 9633) | C/T | M-T; N-C |
| 4 | 3′UTR (POS. 9958) | C/G | N-G; M-C |

M = Mutant huntingtin allele;
N = Normal huntingtin allele.

### Example 3

### An Alternative Method for Determining the Linkage of a Specific SNP Nucleotide to the Disease Associated Allele in HD

The following example describes a novel method for determining the presence of a specific SNP nucleotide in the disease-associated allele in HD. RISC complexes are preloaded with siRNA specific for a SNP nucleotide present in the 3′ region of the htt gene according to art recognized methods. mRNA from a patient with HD, that to heterozygous for the 3′ SNP, is isolated and added to the SNP-specific RISC complexes in vitro and subject to RNAi. The htt mRNA species containing the specific SNP nucleotide targeted by the SNP-specific RISC complex is cleavage into 2 parts whereas the other allele is not. The RNA is then treated with Tobacco Acid Pyrophosphatase to remove the 5′CAP and circularized by treatment with RNA ligase. A region of the circular htt RNA species is amplified by PCR using primers which flank exon 1 (which contains the expanded CAG repeat is HD) and the site of ligation. This PCR product is then sequenced to establish the presence or absence of the disease-associated CAG repeat expansion. If the sequencing identifies the presence of the disease-associated CAG repeat expansion then it can be concluded that the SNP nucleotide specified by the RISC complex is present in the disease-associated htt allele and can be used as a target for RNAi based therapy for HD. If the disease-associated CAG repeat is absent then it can be concluded that the SNP nucleotide is present in the wild-type allele and cannot be used as a target for RNAi based therapy for HD. Hence the present invention provides a rapid, cost-effective and robust method for determining the linkage of a specific SNP nucleotide to the disease associated allele in HD. Moreover, this technique can be used for determining the linkage of any two known nucleotide variants in a disease associated allele.

### Example 4

### Treatment of an HD Patient Using the Methods of the Invention

The following example describes a novel method for treating of an HD patient using the methods of the invention.

An HD patient is selected, based upon the presence of SNP heterozygosities in the alleles of their htt gene. SNP heterozygosities are identified using standard art recognized methods e.g., PCR amplification and sequencing of the patient's htt gene. The presence of specific SNP nucleotides from any of the SNP heterozygosities present in the mutant htt gene is determined using the methods of the invention as described in Examples 1, 2 and 3. Once the specific SNP nucleotides present in the mutant htt gene are determined, allele-specific RNA silencing agents are generated which specifically target the SNP nucleotides present in the mutant htt gene. The patient is then administered the allele-specific RNA silencing agents such that the expression of the mutant huntingtin protein is reduced and the disease is alleviated.

### EQUIVALENTS

Those skilled in the art will recognize, or be able to ascertain using no more than routine experimentation, many equivalents to the specific embodiments of the invention described herein. Such equivalents are intended to be encompassed by the following claims.

What is claimed:

1. A method for identifying the association between a disease-associated mutation and single nucleotide polymorphism (SNP) within a nucleic acid region comprising,
obtaining a linear derivative of the nucleic acid region wherein the region comprises at least one SNP associated with the region,
connecting the ends of the linear derivative to form a circular species in which the disease-associated mutation and SNP are positioned in closer proximity than the naturally-occurring disease associated mutation and SNP,
producing at least a portion of the circular species containing the disease-associated mutation and SNP and,
detecting the presence of the SNP and disease associated mutation thereby identifying their association within the same nucleic acid region.

2. A method for identifying the association between a disease-associated mutation and SNP nucleotide in an RNA molecule comprising,
contacting the RNA molecule with a RISC complex programmed with a gene silencing agent for the SNP, wherein SNP-specific RNA cleavage is achieved,
connecting the ends of the fragments of cleaved RNA to form a circular species,
producing a portion of the circularized species containing the disease-associated mutation and,
detecting the presence of the SNP and disease-associated mutation thereby identifying their association in the same RNA molecule.

3. The method of claim 1 or 2, wherein the identified SNP association is suitable for targeting using gene silencing for achieving therapy for the disease associated mutation.

4. The method of claim 1, wherein the nucleic acid region is DNA or RNA

5. The method of claim 1, wherein the nucleic acid region is a cDNA region, genomic region, chromosomal region, or fragment thereof.

6. The method of claim 1, wherein the obtaining of the linear derivative of the nucleic acid region is by PCR or RT-PCR amplification.

7. The method of claim 1, wherein the obtaining of the linear derivative of the nucleic acid region is obtained by chemical, physical, or enzymatic cleavage of the nucleic acid region.

8. The method of claim 1 or 2, wherein the connecting of the ends of the circular species is by enzymatic ligation.

9. The method of claim 1 or 2, wherein the producing of a portion of the circular species to is achieved by PCR or RT-PCR.

10. The method of claim 1 or 2, wherein the detecting of the presence of the SNP nucleotide is achieved by nucleic acid sequencing.

11. The method of claim 1 or 2, wherein the detecting of the presence of the SNP nucleotide is achieved by nucleic acid hybridization or chip based affinity hybridization.

12. The method of claim 1 or 2, wherein the contacting with a RISC complex is performed in vitro using a cellular extract from Drosophila.

13. The method of claim 2, wherein the RNA is mRNA

14. The method of claim 2, wherein RNA is obtained by reverse transcription from DNA

15. The method of claim 2, wherein producing said portion of said circularized RNA is achieved by RT-PCR

16. The method of claim 1 or 2, wherein said disease-associated mutation is a dominant, gain-of-function mutation.

17. The method of claim 1 or 2, wherein said disease-associated mutation is an oncogenic mutation.

18. The method of claim 1 or 2, wherein said disease-associated mutation comprises an expanded trinucleotide repeat region.

19. The method of claim 1 or 2, wherein disease-associated mutation causes a disease selected from the group consisting of Huntington's disease, spino-cerebellar ataxia type 1, spino-cerebellar ataxia type 2, spino-cerebellar ataxia type 3, spino-cerebellar ataxia type 6, spino-cerebellar ataxia type 7, spino-cerebellar ataxia type 8, spino-cerebellar ataxia type 12, fragile X syndrome, fragile XE MR, Friedreich ataxia, myotonic to dystrophy, spinal bulbar muscular disease and dentatoiubral-pallidoluysian atrophy.

20. The method of claim 1 or 2, wherein the disease is Huntington's disease.

21. The method of any of the above claims for diagnosing a subject having or at risk for a disease arising from a disease-associated mutation.

22. The method of claim 3, wherein therapy is achieved by specifically targeting the disease-associated mutation of an allelic polymorphism encoding a mutant protein.

23. The method of claim 22, wherein the cognate wild type allele of the allelic polymorphism encoding a wild type protein is correctly expressed.

24. A kit for carrying out the methods of any one of the above preceding claims.

25. The kit of claim 24, wherein the kit comprises SNP sequence information or SNP nucleic acid suitable for targeting a disease associated mutation and instructions for use.

26. Use of SNP information or SNP nucleic acid sequence as disclosed herein or as identified according to any of the preceding methods for use in a kit, pharmaceutical composition, research, diagnosis, or therapy.

27. A method for identifying a patient or patient subpopulation amenable to SNP-targeted RNAi therapy wherein the patient or patient subpopulation is first identified as in need of such therapy according to the methods of any one of the preceding claims.
